(19) United States
(12) Patent Application Publication
(10) Pub. No.: US 2014/0018892 A1
(43) Pub. Date: Jan. 16, 2014

(54) IMPLANTABLE MEDICAL LEAD

(71) Applicant: Kenneth Dahlberg, Stockholm (SE)

(72) Inventor: Kenneth Dahlberg, Stockholm (SE)

(73) Assignee: ST. JUDE MEDICAL AB, Jarfalla (SE)

(21) Appl. No.: 13/550,292

(22) Filed: Jul. 16, 2012

(51) Int. Cl. A61N 1/05

(52) U.S. Cl. CPC A61N 1/0573
USPC 607/116

(57) ABSTRACT

An implantable medical lead comprises an outer lead package with an outer insulating tubing and a lead header having a window in its lateral surface. An inner lead package is at least partly arranged in lumens of the outer insulating tubing and the lead header and comprises a helical fixation element connected to a connector pin by an inner conductor coil, and a ring electrode connected to a connector ring by an outer conductor coil. An inner insulating tubing is coaxially arranged between the inner and outer conductor coils. The inner lead package is rotatable relative to the outer lead package and the ring electrode is configured to be at least partly exposed through the window.

### TECHNICAL FIELD

The present embodiments generally relate to implantable medical leads, and in particular to implantable medical leads with improved torque transferring ability.

### BACKGROUND

Magnetic Resonance Imaging (MRI) is of great use for generating an image of the internal tissues of a human body. However, for persons who have an implantable medical lead implanted in their body, there might be problems with induced currents in the implantable medical lead causing, in turn, heating of the lead, in particular at the distal tip of the lead.

In vitro MRI experiments have shown that an implantable medical lead acts like an antenna since the effective length of the lead is close to a multiple of the radio frequency (RF) wavelength of the MRI scanning equipment and thereby receives the pulsed RF signal of the MRI scanning equipment. The reception of the RF energy results in an RF wave propagating along the lead and heating the lead tip to an unacceptable level. Some other parts of the lead become heated as well.

Various solutions to combat this MRI-dependent problem for implantable medical leads have been suggested in the art. U.S. Published Application Nos. 2011/0125240 and 2011/0301676 disclose solutions to the problem by arranging a tip inductor between the helical fixation electrode and the inner conductor coil and a ring inductor between the ring electrode and the outer conductor coil. These prior art implantable medical leads work well in connection with MRI scanning but the inclusion of the inductors and in particular the ring inductor makes manufacture of the implantable medical lead a complex process requiring a high level of skill of the manufacturer.

A further limitation of prior art MRI-compatible leads and in particular such leads with a distributed inductance is low torque transferring ability when screwing in/out the helical fixation electrode.

U.S. Published Application No. 2002/0183820 discloses a lead assembly including a lead body comprising one or more conductors therein. A braided torque transmission member is included in the lead assembly and is rotatable to extend and/or retract a helical fixation element.

U.S. Published Application No. 2007/0179582 discloses a coil conductor for connecting an electrode of a medical electrical lead with an implantable medical device. The coil conductor includes a multi-filar coil and a torque enhancing sheating. The multi-filar coil comprises a co-radially wound, multi-filar coil that has an inductance of at least 1.5 pH. The sheating is extruded over the multi-filar coil to enhance the torque transmitting properties of the coil conductor.

U.S. Pat. No. 8,112,160 discloses a lead with a lead body, a helical composite electrode, a composite conductor and a proximal connector. The helical composite electrode has a first electrode and a second electrode in a co-axial configuration. The composite conductor electrically connects the first and second electrode to the proximal connector.

### SUMMARY

An aspect of the embodiments relates to an implantable medical lead comprising an outer lead package and an inner lead package.

The outer lead package comprises an outer insulating tubing running from a proximal lead portion to a distal lead portion of the implantable medical lead and has a lumen. The outer lead package also comprises a lead header made of an electrically insulating material and comprising at least one window provided in a lateral surface of the lead header. The lead header has a lumen and is joined to a distal end of the outer insulating tubing.

The inner lead package comprises a helical fixation element electrically connected to a connector pin by an inner conductor coil, and a ring electrode electrically connected to a connector ring by an outer conductor coil. The connector pin and the connector ring are connectable to an implantable medical device. The inner lead package also comprises an inner insulating tubing coaxially arranged relative to and between the outer conductor coil and the inner conductor coil.

The inner lead package is at least partly arranged in the lumen of the outer insulating tubing and the lumen of the lead header. The inner lead package is rotatable relative to the outer lead package. The ring electrode of the inner lead package is configured to be at least partly exposed through the at least one window of the outer lead package.

The embodiments significantly improve the torque transferring ability by providing an inner lead package with two conductor coils and an inner insulating tubing that is rotatable relative to the outer lead package in order to move the helical fixation element from a retracted state in the lumen of the lead header to an extended state with the helical fixation element protruding beyond the distal end of the lead header.

This high torque transferring ability is possible even in connection with MRI compatible leads generally requiring inner conductor coils of high inductance and thereby having few conductor filars.

### BRIEF DESCRIPTION OF THE DRAWINGS

The invention, together with further objects and advantages thereof, may best be understood by making reference to the following description taken together with the accompanying drawings, in which:

FIG. 1 is a schematic overview of a human subject having an implantable medical lead according to an embodiment connected to an implantable medical device to provide cardiac sensing and/or therapy;

FIG. 2 is a schematic overview of an implantable medical lead according to an embodiment connectable to an implantable medical device;

FIG. 3 is a cross-sectional view of a lead body of an implantable medical lead according to an embodiment;

FIG. 4 illustrates a proximal lead portion according to an embodiment;

FIG. 5 is a partly cross-sectional view of the proximal lead portion shown in FIG. 4;

FIG. 6 illustrates a distal portion of an outer lead package of an implantable medical lead according to an embodiment;

FIG. 7 illustrates a distal portion of an inner lead package of an implantable medical lead according to an embodiment;

FIG. 8a illustrates a distal lead portion of an implantable medical lead prior to rotation of an inner lead package relative to an outer lead package; and

FIG. 8b illustrates a distal lead portion of an implantable medical lead following rotation of an inner lead package relative to an outer lead package.

### DETAILED DESCRIPTION

Throughout the drawings, the same reference numbers are used for similar or corresponding elements.

The present embodiments generally relate to implantable medical leads, and in particular to such implantable medical leads that are of the active fixation type. The implantable medical leads of the embodiments have improved torque transferring ability and can advantageously be designed to be MRI compatible.

In particular, the embodiments solve a prior art problem of MRI compatible implantable medical leads. An implantable medical lead can be designed to be MRI compatible by presenting a certain level of inductance. A distributed inductance over a substantial portion of the lead length is generally preferred over discrete inductors due to cost issues. In such a case, the inner conductor coil of the implantable medical lead functions as an inductor. In order to achieve a sufficiently high level of inductance the inner conductor coil is preferably designed to comprise many turns of a wire wound to form the inner conductor coil. Providing many turns and thereby a high inductance generally implies few conductor filars. However, such an inner conductor coil with a distributed inductance typically has low torque transfer ability. This might make it more difficult to anchor the helical fixation element of the implantable medical lead at a target tissue during implantation. Hence, there will generally be a conflict between sufficiently high inductance and thereby MRI compatibility of the implantable medical lead and sufficiently high torque transfer ability in prior art implantable medical leads.

The present embodiments present an implantable medical lead that can be designed to have high level of inductance and thereby be MRI compatible and still have sufficient torque transfer ability to efficiently anchor the implantable medical lead in a target tissue in a subject during implantation.

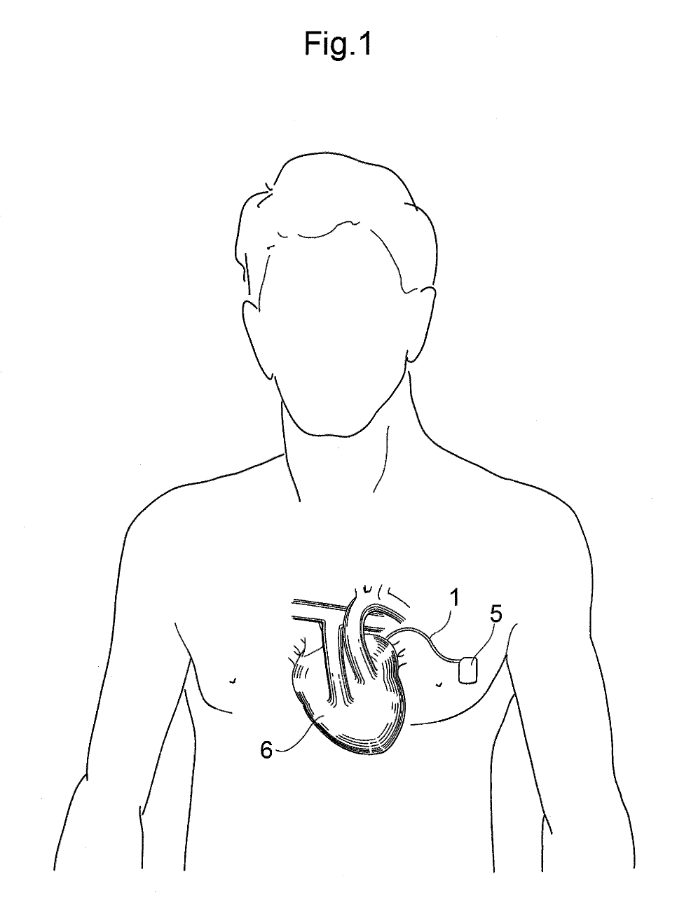

FIG. 1 is a schematic overview of a mammalian subject, represented by a human subject, having an implantable medical lead 1 according to the embodiments implanted in the body. In FIG. 1 the implantable medical lead 1 interconnects an implantable medical device (IMD) 5, such as a pacemaker or an implantable cardioverter-defibrillator (ICD), and a target tissue, here represented by a heart 6. Hence, the implantable medical lead 1 comprises electrodes arranged in connection with its distal portion that are used for sensing electrical activity of the heart 6 by the IMD 5 and/or delivering pacing pulses or defibrillation shocks generated by the IMD 5 to the heart 6.

The implantable medical lead 1 of the embodiments is advantageously used in connection with cardiac therapy or diagnosis and is thereby provided in or in connection with a subject's heart 6. The embodiments are, however, not limited thereto and the implantable medical lead can 1 in fact be used to provide electrical sensing and/or therapy delivery to any tissue or organ in the subject body at which the implantable medical lead 1 can be anchored by screwing its helical fixation element into or in the vicinity of the relevant tissue or organ.

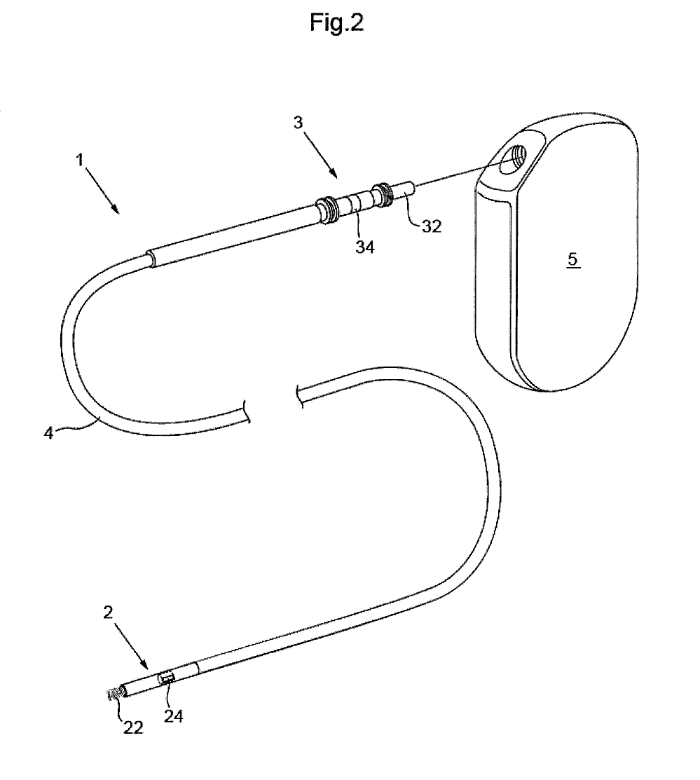

FIG. 2 is a schematic overview of an implantable medical lead 1 according to an embodiment connectable to an IMD 5. The implantable medical lead 1 comprises a proximal lead portion 3 configured to be connected to the IMD 5. This proximal lead portion 3 comprises a connector pin 32 and a connector ring 34 that are connectable to the IMD 5 and to matching electrode terminals (not shown) provided in the IMD 5. An opposite, distal lead portion 2 of the implantable medical lead 1 comprises electrodes 22, 24 that are to be arranged at the tissue or organ in the subject body in order to provide electrical sensing and/or therapy. According to the embodiments, the electrodes 22, 24 comprise a helical fixation element 22 that is designed in the form of a screw and is configured to be screwed into a tissue of the subject's body to anchor the helical fixation element 22 and thereby the implantable medical lead 1 at the tissue.

The helical fixation element 22 is electrically connected to the connector pin 32 in the proximal lead portion 3 by an inner conductor coil running in a lead body 4 of the implantable medical lead 1 extending from the proximal lead portion 3 to the distal lead portion 2.

The other electrode is in the form of a ring electrode 24 that is electrically connected to the connector ring 34 by an outer conductor coil running in the lead body 4.

In FIG. 2 the implantable medical lead 1 has been illustrated as a bipolar implantable medical lead 1, i.e. having two sensing/pacing electrodes 22, 24. The embodiments are, however, not limited thereto but can be applied to an implantable medical lead 1 having a helical fixation electrode 22 and at least one ring electrode 24. Thus, the embodiments can also be applied to so-called tripolar or quadropolar implantable medical leads or indeed implantable medical leads having two, three or even more ring electrodes 24 in addition to the helical fixation element 22.

An aspect of the embodiments relates to an implantable medical lead comprising an outer lead package and an inner lead package.

The outer lead package comprises an outer insulating tubing running from a proximal lead portion to a distal lead portion of the implantable medical lead. The outer insulating tubing has a lumen. The outer lead package also comprises a lead header made of an electrically insulating material and comprises at least one window provided in a lateral surface of the lead header. The lead header has a lumen and is joined or connected to a distal end of the outer insulating tubing.

The inner lead package comprises a connector pin that is connectable to an IMD, a helical fixation element and an inner conductor coil having a proximal end electrically connected to the connector pin and a distal end electrically connected to the helical fixation element. The inner lead package also comprises a connector ring connectable to the IMD, a ring electrode and an outer conductor coil having a proximal end electrically connected to the connector ring and a distal end electrically connected to the ring electrode. The outer conductor coil and the inner conductor coil are coaxially arranged relative to each other with the inner conductor coil in the lumen of the outer conductor coil. An inner insulating tubing of the inner lead package is coaxially arranged relative to and between the outer conductor coil and the inner conductor coil to electrically insulate the conductor coils from each other.

According to the embodiments, the inner lead package is at least partly arranged in the lumen of the outer insulating tubing and the lumen of the lead header. Furthermore, the inner lead package is rotatable relative to the outer lead package. Rotation of the inner lead package relative to the outer lead package will at least partly expose the ring electrode of the inner lead package through the at least one window in the lead header of the outer lead package.

Hence, according to the embodiments the complete inner lead package, comprising not only the inner conductor coil but also the outer conductor coil and the intermediate inner insulating tubing together with the electrodes (helical fixation element and ring electrode) and electrode terminals (connector pin and connector ring) of the inner and outer conductor coils, is rotatable relative to the outer lead package. This provides a significant higher torque transfer ability as compared to only having the inner conductor coil with the connector pin and helical fixation element rotatable relative to the outer insulating tubing, the lead header, the outer conductor coil and the inner insulating tubing according to prior art lead designs.

The embodiments thereby provide two distinct lead portion assemblies with the outer lead package acting as a stator and the inner lead package as the rotor. The torque transferring assembly of the embodiments comprises both conductor coils and the intermediate inner insulating tubing and thereby presents a high torque transferring ability.

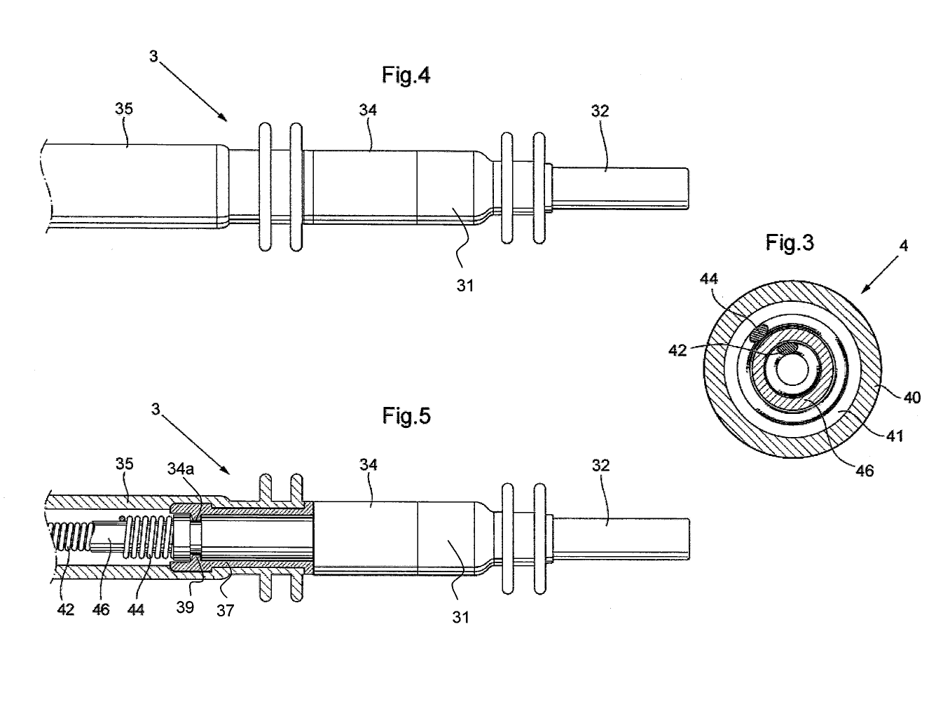

FIG. 3 is a cross-sectional view of the lead body 4 of an implantable medical lead according to an embodiment. The outer portion of the lead body 4 is formed by the outer insulating tubing 40 that has a lumen 41 in which at least part of the inner lead package is arranged. FIG. 3 illustrates the outer conductor coil 44, the inner conductor coil 42 and the intermediate inner insulating tubing 46 of the inner lead package. This part of the inner lead package is thereby arranged in the lumen 41 of the outer insulating tubing 40 and is rotatable relative to the outer insulating tubing 40.

FIG. 4 is a view of the proximal lead portion 3 of the implantable medical lead. FIG. 4 illustrates the connector pin 32 of the inner lead package that is connectable to the IMD and is electrically connected to the helical fixation element in the distal lead portion by the inner conductor coil running in the lumen of the outer insulating tubing. FIG. 4 also illustrates the connector ring 34 that is connectable to the IMD and is electrically connected to the ring electrode in the distal lead portion by the outer conductor coil. Reference number 35 indicates an optional connector boot 35, to which the outer insulating tubing can be attached, such as adhesively attached by a medical grade adhesive, such as silicone adhesive. A front seal 31 is typically provided as a part of the inner lead package and is made of an electrically insulating material. The front seal 31 is advantageously interposed between the connector pin 32 and the connector ring 34. The front seal 31 is designed to prevent or at least inhibit entry of blood into the lead connecting part of the IMD (see entrance in the IMD 5 in FIG. 2) when the connector pin 32, the front seal 31 and the connector ring 34 are introduced into the lead connecting part of the IMD.

FIG. 5 is a partly cross-sectional view of the proximal lead portion 3 shown in FIG. 4. FIG. 5 shows the outer conductor coil 44 having its proximal end, directly or indirectly, electrically connected to the connector ring 34. The inner conductor coil 42 with its proximal end, directly or indirectly, electrically connected to the connector pin 32 is also indicted in FIG. 5 together with the inner insulating tubing 46 coaxially arranged between the outer conductor coil 44 and the inner conductor coil 42.

In a particular embodiment the outer lead package comprises an electrically insulating insert 37 of or joined to the connector boot 35. The electrically insulating insert 37 has a flange structure 39 configured to protrude into a circular slot 34a of the connector ring 34. The flange structure 39 forms together with the circular slot 34a a rotatable connection between the outer lead package and the inner lead package in the proximal lead portion 3.

This rotatable connection could alternatively be provided by having a circular recess (not shown) in the electrically insulating insert 37 into which a flange structure (not shown) of the connector ring 34 is configured to protrude.

These two embodiments work equally well and both provide a rotatable connection between the outer lead package and the inner lead package in the proximal lead portion.

The embodiments are not limited to the above-mentioned rotatable connections but also encompass alternative embodiments of providing such a rotatable connection in the proximal lead portion.

In FIG. 5 the connector ring 34 comprises a cylindrical ring portion that is electrically connected to a matching connector terminal in the IMD and an axial portion running in the lumen of the electrically insulating insert 37. This axial portion comprises the circular slot 34a or the flange structure that engages the flange structure 39 or the circular recess of the electrically insulating insert 37 to form the rotatable connection.

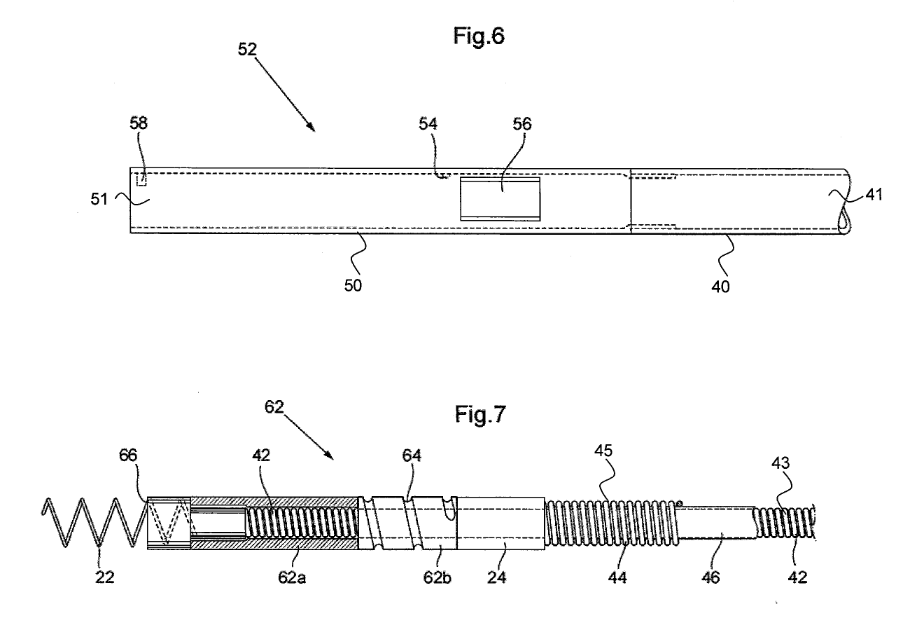

FIG. 6 illustrates a distal portion of the outer lead package 52 according to an embodiment. The distal portion comprises the distal end of the outer insulating tubing 40 running along the lead body of the implantable medical lead and up to the proximal lead portion and the optional connector boot. The distal end of the outer insulating tubing 40 is attached to the lead header 50, such as adhesively attached to the lead header 50. FIG. 6 also shows the respective lumens 51, 41 of the lead header 50 and the outer insulating tubing 40 in which the inner lead package is at least partly arranged.

The lead header 50 comprises, as previously mentioned, at least one window 56 provided in a lateral or envelope surface of the lead header 50. The at least one window 56 is arranged in the lead header 50 to expose at least a portion of the ring electrode of the inner lead package when the helical fixation element and the ring electrode have been moved relative to the outer lead package 52 to an extended state with the helical fixation element at least partly extending beyond a distal end of the lead header 50.

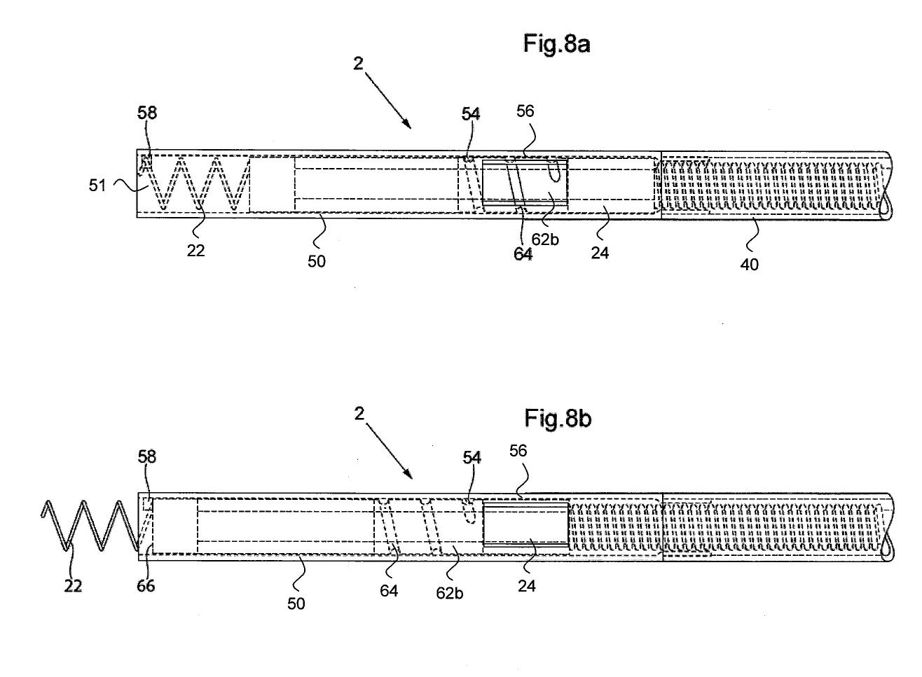

Thus, during implantation the helical fixation element 22 and the ring electrode 24 are present in a retracted state relative to the outer lead package 52 and the helical fixation element 22 is preferably present in the lumen 51 of the lead header 50, see FIG. 8a. In this retracted state the helical fixation element 22 preferably does not extend beyond the distal end of the lead header 50, which otherwise could cause damages to blood vessels and other tissues during implantation of the implantable medical lead when it is being moved towards the intended implantation site. Once the implantation site is reached the inner lead package is rotated by the physician relative to the outer lead package 52. This rotation is transformed, which is further described herein, into a translational or longitudinal movement of the helical fixation element 22 and the ring electrode 24 relative to the outer lead package 52, see FIG. 8b. This means that the helical fixation element 22 and the ring electrode 24 are moved from the retracted state to the above-mentioned extended state.

In the retracted state the helical fixation element 22 is preferably present in the lumen 51 of the lead header 50 and the ring electrode 24 is typically present in the lumen 51 of the lead header 50 or indeed at least partly in the lumen 41 of the outer insulating tubing 40. At this position the ring electrode 24 or at least a portion thereof will not be exposed through the at least one window 56 but rather be present proximally relative to the at least one window 56.

In the extended state at least a portion of the helical fixation element 22 is moved out of the distal end of the lead header 50 to be screwed into a target tissue. The ring electrode 24 is also moved in the extended state towards the distal end of the lead header 50 and will thereby be aligned with the at least one window 56 and is therefore exposed and accessible through the at least one window 56. Thus, at the extended state the ring electrode 24 is exposed to enable electrical sensing from surrounding tissue and/or delivery of pacing pulses to surrounding tissue through the at least one window 56.

In FIG. 6 a single window 56 is provided in the lateral surface of the lead header 50. This is generally sufficient and enables access to the ring electrode in the extended state for surrounding tissue. However, in such an approach the electrical sensing and/or pacing performed by the ring electrode through the window 56 is generally directed towards the particular tissue situated close to the window 56. It might then be necessary to confirm, following implantation, that the window 56 and thereby the ring electrode is facing the intended tissue in the subject body.

In an alternative approach the lead header 50 comprises multiple windows 56 in its lateral surface, such as two, three or more windows 56. In such a case, these windows 56 are preferably arranged at substantially a same distance in the lateral surface relative to the distal end of the lead header 50. The windows 56 are preferably further substantially uniformly distributed around the circumference of the lead header 50. For instance, if two or three windows 56 are provided their centers are preferably provided at an angle of about 180° or 120° between adjacent windows as seen from a cross-sectional view of the lead header 50 as taken in a plane with a normal parallel to the longitudinal axis of the lead header 50.

Having two, three or indeed more windows 56 imply that the ring electrode will be exposed substantially all around the circumference of the lead header 50 and the need for rotating a single window 56 and the ring electrode to face a target tissue is thereby reduced.

The lead header 50 preferably comprises at least one guide structure 54, 58 radially protruding into the lumen 51 of the lead header 50. This guide structure 54, 58 is then configured to engage at least a portion of the inner lead package to transform a rotation of the inner lead package relative to the outer lead package 52 into a translational movement of the helical fixation element and the ring electrode relative to the outer lead package 52. FIG. 6 illustrates two different embodiments of such a guide structure 54, 58.

In an embodiment the guide structure is in the form of a post 58 radially protruding into the lumen 51 of the lead header 50. The post 58 is then configured to be interposed between adjacent turns of the helical fixation element. At this position rotation of the inner lead package relative to the outer lead package 52 will be transformed by the post 58 into a longitudinal movement of the helical fixation element and thereby also of the ring electrode relative to the outer lead package 52.

The post 58 can be an integrated part of the lead header 50 thereby arranged in the inner surface of the lead header 50 to protrude into the lumen 51. Alternatively, the lead header 50 comprises a through-hole into which a post structure is inserted to have a portion extending into the lumen 51. In either case, the post 58 is typically arranged in the vicinity of the distal end of the lead header 50 as shown in FIG. 6.

In an alternative embodiment, the inner lead package 62 comprises a groove member 62b, see FIG. 7, having an outer surface comprising a helical groove 64. The guide structure 54 is then configured to engage the helical groove 64 to transform a rotation of the inner lead package 62 relative to the outer lead package 52 into a translational movement of the helical fixation element 22, the groove member 62b and the ring electrode 24 relative to the outer lead package 52.

The guide structure 54 can in an embodiment be in the form of a guide pin interacting with the helical groove 64. The guide pin is then arranged in the inner surface of the lead header 50 and could be an integrated part of the lead header 50 or is inserted into a through-hole in the lead header 50 as discussed above for the post 58.

In an alternative embodiment, the guide structure 54 is in the form of a screw thread provided on a portion of the inner surface of the lead header 50. The screw thread then engages the helical groove 64 of the groove member 62b to achieve the desired rotation-to-translation transformation.

In an embodiment, the lead header 50 comprises the guide structure 58. In an alternative embodiment the lead header 50 comprises the guide structure 54, or in another embodiment the lead header 50 comprises both the guide structure 54 and the guide structure 58.

FIG. 7 illustrates a distal portion of the inner lead package 62. In the illustrated embodiment the helical fixation element 22 is electrically and mechanically connected to the inner conductor coil 42 through an optional helix base 66. This helix base 66 thereby provides a mechanical and electrical bridge between a proximal end of the helical fixation element 22 and a distal end of the inner conductor coil 42. The inner conductor coil 42 could be attached to the helix base 66 though, for instance, welding or crimping.

If the lead header 50 comprises a post 58 as shown in FIG. 6, the distal end of the helix base 66 could abut the post 58 when the helical fixation element 22, the helix base 66 and the ring electrode 24 have been moved relative to the outer lead package 52 to the extended state. Hence, the abutment of the helix base 66 and the post 58 provides an effective stop of the rotation of the inner lead package 62 relative to the outer lead package 52 and a stop of the translational movement of the helical fixation element 22, the helix base 66 and the ring electrode 24 relative to the outer lead package 52.

In an optional embodiment, the cylindrical space between the helix base 66 and the ring electrode 24 or the optional groove member 62b is filled with or occupied by a cylinder 62a of an electrically insulating material, such as silicone or polyether ether ketone (PEEK). The cylinder 62a thereby functions as a spacer to provide a target distance between the ring electrode 24/the optional groove member 62b and the helical fixation element 22/the helix base 66.

In a particular embodiment, the inner conductor coil 42 is attached to the connector pin 32 and to the helix base 66 by welding or crimping. The inner insulating tubing 46, for instance made of silicone, polyurethane or a combination thereof, could be adhesively attached to the connector pin 32 and the helix base 66. The previously mentioned front seal 31 and the connector ring 34 are preferably attached to the connector pin 32. The outer conductor coil 44 is attached to the connector ring 34 and the ring electrode 24, such as by welding or crimping. The ring electrode 24 is preferably attached at the distal end of the inner insulating tubing 46. The cylindrical space between the helix base 66 and the ring electrode 24 may be filled with a cylinder 62a of an insulating material. In an embodiment, these elements form part of the inner lead package 62.

In a particular embodiment, the inner insulating tubing 46 is mechanically attached to the inner conductor coil 42 and to the outer conductor coil 44 at multiple locations or sites along the lead body 4. The attachment can, for instance, be made with an adhesive, preferably silicon adhesive. In a preferred embodiment, the attachment sites are spaced apart along the lead body 4. These spot wise attachments will increase the torque transfer ability of the inner lead package 62 without substantially increasing the stiffness of the inner lead package 62.

The outer insulating tubing 40, for instance made of silicone, polyurethane or a combination thereof, is preferably adhesively attached to the distal end of the connector boot 35 and to the lead header 50, which may be of for instance PEEK or another electrically insulating polymer or plastic material. The lead header 50 preferably comprises at least one guide structure 54, 58 as previously disclosed herein. These elements form part, in an embodiment, of the outer lead package 52.

The proximal end of the outer lead package 52 may be assembled on a connector part of the inner package, such that the electrically insulating insert 37 of the connector boot 35 slides in a circular slot 34a in the connector ring 34.

The above mentioned parts of the implantable medical lead can be manufactured using process methods and materials well known in the art.

FIG. 8a illustrates a distal lead portion 2 of the implantable medical lead prior to rotation of the inner lead package relative to the outer lead package, i.e. the retracted state, whereas FIG. 8b illustrates the distal lead portion 2 following rotation of the inner lead package relative to the outer lead package, i.e. the extended state.

As shown in FIG. 8a, in the retracted state the helical fixation element 22 is preferably fully present in the lumen 51 of the lead header 50 or at least a major part of the helical fixation element 22 is inside the lumen 51. In the embodiment shown in FIG. 8a, the ring electrode 24 is arranged in the inner lead package at a position beyond the at one least window 56 in the lead header 50, i.e. positioned proximally relative to the window 56. In the retracted state shown in FIG. 8a, the groove member 62b with its helical groove 64 is at least partly aligned with the at least one window 56.

When the inner lead package is rotated relative to the outer lead package, typically by rotating the connector pin in the proximal lead portion while holding the outer insulating tubing 40 or the connector boot, the helical fixation element 22 will be moved out from the distal end of the lead header 50 and the ring electrode 24 is moved to be aligned with and exposed through the at least one window 56. The rotation of the inner lead package relative to the outer lead package is preferably performed until the distal end of the helix base 66 abuts the post 58 and/or the guide structure 54 reaches the proximal end of the helical groove 64 of the groove member 62b.

Entry of blood into the lumen 51 of the lead header 50 can be minimized by designing the outer diameter of the portion of the inner lead package aligned with the at least one window 56 in the retracted state and the extended state (typically the groove member 62b and the ring electrode 24) to be substantially equal to or only slightly smaller than the diameter of the lumen 51 of the lead header 50. Such a design will reduce the amount of blood that can leak through the window 56.

Alternatively or in addition one or more blood seals in the form of, for instance, a silicone ring can be arranged in connection with the ring electrode 24. This effectively prevents blood from entering the lumen of the outer insulating tubing 40 and the lumen 51 of the lead header 50. A further variant of a blood seal is to provide a silicone frame around the at least one window 56 to engage the outer surface of the inner lead package, such as groove member 62b and ring electrode 24, and thereby form a tight seal that inhibits any entry of blood.

As indicated in FIG. 7 the inner conductor coil 42 is preferably formed of a first wire 43 having an electrically conductive core and an outer electrically insulating coating. This first wire 43 is then wound in a given winding direction. The outer conductor coil 44 is correspondingly preferably formed of a second wire 45 having an electrically conductive core and an outer electrically insulating coating. The second wire 45 is preferably wound in the given winding direction. Hence, the two conductor coils 42, 44 preferably have the same winding direction. This improves the radio frequency (RF) attenuation of the implantable medical lead in an MRI environment. The winding direction may be left hand wound or right hand wound.

The electrically conductive core of the first wire 43 and optionally of the second wire 45 is preferably made of an electrically conductive material selected from a group consisting of tantalum, niobium and a silver filled nickel-cobalt-chromium-molybdenum alloy, such as silver filled MP35N®. The outer electrically insulating coating of the first wire 43 and the second wire 45 can be made from well known insulating materials, such as ethylene tetrafluoroethylene (ETFE), polytetrafluoroethylene (PTFE), perfluoroalkoxy (PFA), polyimide, polyurethane, etc.

In a particular embodiment, the inner conductor coil 42 is of a few filar design in order to maximize the inductance. The inner conductor coil 42 consequently preferably comprises no more than three filars, such as two filars or a single filar. In this way the number of turns of the inner conductor coil 42 increases and the inductance is substantially increased. The inner conductor coil 42 preferably has an inductance in parity with or preferably higher than the inductance of the outer conductor coil 44.

The outer conductor coil 44 is preferably a multifilar conductor comprising at least five filars. In this way the inductance of the outer conductor coil 44 is sufficiently high without being substantially higher than the inductance of the inner conductor coil 42.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. An implantable medical lead comprising:
an outer lead package comprising:
an outer insulating tubing running from a proximal lead portion to a distal lead portion and having a lumen; and
a lead header made of an electrically insulating material and having a header lumen; and
an inner lead package comprising:
a connector pin connectable to an implantable medical device;
a helical fixation element;
an inner conductor coil having a proximal end electrically coupled to said connector pin and a distal end electrically coupled to said helical fixation element;
a connector ring connectable to said implantable medical device;
a ring electrode;
an outer conductor coil having a proximal end electrically connected to said connector ring and a distal end electrically connected to said ring electrode; and
an inner insulating tubing coaxially arranged relative to and between said outer conductor coil and said inner conductor coil, wherein said inner lead package is at least partly arranged in said lumen of said outer insulating tubing and said header lumen, and said inner lead package is rotatable relative to said outer lead package to translate the fixation element relative to the fixed outer lead package.

2. The implantable medical lead according to claim 1, wherein the lead header includes at least one window provided in a lateral surface of said lead header and wherein said lead header is joined to a distal end of said outer insulating tubing and wherein said lead header comprises a guide structure radially protruding into said lumen of said lead header, said guide structure is configured to engage at least a portion of said inner lead package to transform a rotation of said inner lead package relative to said outer lead package into a translational movement of said helical fixation element and said ring electrode relative to said outer lead package.

3. The implantable medical lead according to claim 2, wherein said ring electrode is arranged in said inner lead package at a position that is aligned with said at least one window when said helical fixation element and said ring electrode have been moved relative to said outer lead package to an extended state with at least a portion of said helical fixation element extending beyond a distal end of said lead header.

4. The implantable medical lead according to claim 2, wherein said ring electrode is arranged in said inner lead package at a position beyond said at least one window when said helical fixation element and said ring electrode are in a retracted state relative to said outer lead package with said helical fixation element present in said header lumen.

5. The implantable medical lead according to claim 2, wherein said guide structure is in the form of a post radially protruding into said lumen of said lead header, said post is configured to be interposed between adjacent turns of said helical fixation element.

6. The implantable medical lead according to claim 5, wherein said inner lead package comprises a helix base electrically and mechanically interconnecting a proximal end of said helical fixation element with said distal end of said inner conductor coil, a distal end of said helix base is configured to abut said post when said helical fixation element, said helix base and said ring electrode have been moved relative to said outer lead package to an extended state with at least a portion of said helical fixation element extending beyond a distal end of said lead header.

7. The implantable medical lead according to claim 2, wherein said inner lead package comprises a groove member having an outer surface comprising a helical groove, said guide structure is configured to engage said helical groove to transform said rotation of said inner lead package relative to said outer lead package into said translational movement of said helical fixation element, said groove member and said ring electrode relative to said outer lead package.

8. The implantable medical lead according to claim 1, wherein said lead header comprises three windows arranged at a same distance in said lateral surface relative to a distal end of said lead header and uniformly distributed around a circumference of said lead header.

9. The implantable medical lead according to claim 1, wherein said inner conductor coil is formed of a first wire having an electrically conductive core and an outer electrically insulating coating wound in a winding direction and said outer conductor coil is formed of a second wire having an electrically conductive core and an outer electrically insulating coating wound in said winding direction.

10. The implantable medical lead according to claim 9, wherein said electrically conductive core of said first wire is made of an electrically conductive material selected from a group consisting of tantalum, niobium and silver filled nickel-cobalt-chromium-molybdenum alloy.

11. The implantable medical lead according to claim 1, wherein said inner lead package comprises a helix base electrically and mechanically interconnecting a proximal end of said helical fixation element with said distal end of said inner conductor coil, a proximal end of said inner insulating tubing is adhesively attached to said connector pin and a distal end of said inner insulating tubing is adhesively attached to said helix base.

12. The implantable medical lead according to claim 1, wherein said inner lead package comprises a front seal of an electrically insulating material interposed between said connector pin and said connector ring.

13. The implantable medical lead according to claim 1, wherein said outer lead package comprises an electrically insulating insert having a flange structure configured to protrude into a circular slot of said connector ring to form a rotatable connection between said outer lead package and said inner lead package in said proximal lead portion.

14. The implantable medical lead according to claim 1, wherein said outer lead package comprises an electrically insulating insert having a circular recess into which a flange structure of said connector ring is configured to protrude to form a rotatable connection between said outer lead package and said inner lead package in said proximal lead portion.

15. The implantable medical lead according to claim 1, wherein said inner conductor coil comprises no more than three filars.

16. The implantable medical lead according to claim 1, wherein said outer conductor coil is a multifilar conductor coil comprising at least five filars.

17. The implantable medical lead according to claim 1, wherein said inner insulating tubing is attached to said inner conductor coil and to said outer conductor coil at multiple attachment sites spaced apart along a length of said inner insulating tubing.

18. The implantable medical lead according to claim 17, wherein said inner insulating tubing is attached to said inner conductor coil and to said outer conductor coil at said multiple attachment sites by an adhesive.

19. An implantable medical lead comprising:
an outer insulating tubing running from a proximal lead portion to a distal lead portion and having a lumen;
an inner lead package comprising
a helical fixation element;
an inner conductor coil having a distal end electrically coupled to the helical fixation element;
a ring electrode;
an outer conductor coil having a distal end electrically coupled to the ring electrode; and
an inner insulating tubing coaxially arranged relative to and between the outer conductor coil and the inner conductor coil, wherein said inner lead package is at least partly arranged in said lumen of the outer insulating tubing and the inner lead package is rotatable relative to the outer insulating tubing to translate the fixation element relative to the fixed outer lead package.

20. The implantable medical lead according to claim 19, wherein the inner insulating tubing is attached to the inner conductor coil and to the outer at multiple attachment sites spaced apart along a length of said inner insulating tubing.
